(19) United States
(12) Patent Application Publication
(10) Pub. No.: US 2022/0249937 A1
(43) Pub. Date: Aug. 11, 2022

(54) METHOD AND SYSTEM OF OPTIMIZING AND PERSONALIZING RESISTANCE FORCE IN AN EXERCISE

(71) Applicants: Leonardo von Prellwitz, san francisco, CA (US); Giorgio Duimich, Rome (IT); Andrea Duimich, Rome (IT)

(72) Inventors: Leonardo von Prellwitz, san francisco, CA (US); Giorgio Duimich, Rome (IT); Andrea Duimich, Rome (IT)

(21) Appl. No.: 17/535,339

(22) Filed: Nov. 24, 2021

Related U.S. Application Data
(63) Continuation of application No. 15/599,977, filed on May 19, 2017, now abandoned.
(60) Provisional application No. 62/338,938, filed on May 19, 2016.

(51) Int. Cl. A63B 71/06; A63B 24/00

(52) U.S. Cl. CPC A63B 71/0622; A63B 24/0075; A63B 2220/40; A63B 2230/75; A63B 2225/15; A63B 2225/096; A63B 2225/54; A63B 2230/06; A63B 2220/51; A63B 2024/0065

(57) ABSTRACT

In one aspect, a method useful for automating, personalizing, and optimizing a resistance force in an exercise across time and space, the system including the step of providing an exercise machine. The method includes the step of providing a biometric sensor coupled with a user performing an exercise on the exercise machine. The method includes the step of obtaining a user's profile data, wherein the user's profile data comprises factors such as a user's history of exercising on the exercise machine. The method includes the step of obtaining a user input into an exercise resistance controller of the exercise machine. The method includes the step of, while the user performs one or more repetitions of the exercise on the exercise machine, obtaining real-time data of a set of parameters of the one or more repetitions of the exercise on the exercise machine. The method includes the step of obtaining a user's biometric data.

### CROSS-REFERENCE TO RELATED APPLICATIONS

This application claims priority to U.S. application Ser. No. 15/599,977 filed on May 19, 2017 and titled Method And System Of Optimizing Resistance Force In An Exercise. This application is incorporated by reference in its entirety.

U.S. patent application Ser. No. ______ claims priority to U.S. Provisional Application No. 62/338,938 filed on 19 May 2016 and titled Method And System Of Optimizing Resistance Force In An Exercise. This provisional application is incorporated by reference in its entirety.

### BACKGROUND OF THE INVENTION

### 1. Field

This application relates to exercise machines and more specifically to a system, article of manufacture, and method of optimizing resistance force in an exercise.

### 2. Related Art

With the increasing proliferation of sensor data in the fitness realm and the steadily decreasing cost of electric engines, the fitness industry is ripe for a transition from analog (using weights and other types of analog resistance) to digital and sensor driven. In this sense, there is a need for an entirely new software and firmware architecture to be able to effectively utilize data sources in order to drive the algorithms that steer these machines. Pairing fitness sensor data with an actuator gives the opportunity for the first time to set training data in a feedback loop to allow the system to increase in accuracy with increased use across time and from a larger user set.

### BRIEF SUMMARY OF THE INVENTION

In one aspect, a computing method useful for automating, personalizing, and optimizing a resistance force in an exercise across time and space, the method comprising: providing a personalized training session for a first user and a second user, wherein the personalized training session comprises a work parameter scheme (WPS); providing a first exercise machine, wherein the first exercise machine comprises a first digitally adjustable resistance device; providing a second exercise machine, wherein the second exercise machine comprises a second digitally adjustable resistance device; providing a first biometric sensor coupled with the first user performing an exercise on the first exercise machine; providing a second biometric sensor coupled with the second user performing an exercise on the second exercise machine; obtaining a first user's profile data, wherein the first user's profile data comprises a first user's history of exercising on the first exercise machine; obtaining a second user's profile data, wherein the second user's profile data comprises a second user's history of exercising on the second exercise machine; obtaining a first user input into a first exercise resistance controller of the first exercise machine; obtaining a second user input into a second exercise resistance controller of the second exercise machine; while the first user and the second user perform one or more repetitions of the exercise on the first exercise machine and the second exercise machine, respectively: obtaining a real-time data of a set of parameters of the one or more repetitions of the exercise on the first exercise machine and the second exercise machine, wherein the real-time data is used as a proxy for fatigue in the first user and the second user; obtaining a first user's biometric data; obtaining a second user's biometric data; and based on the real-time data, the first user's biometric data, the second user's biometric data, the first user input into the first exercise resistance controller, the second user input into the second exercise resistance controller, the first user's profile data, the second user's profile data, and the WPS: analyzing the specified data points that act as proxy for fatigue, wherein the specified data points that act as proxy for fatigue comprise an acceleration from a machine sensor and a heart rate from a BRD; analyzing the real-time data with respect to the first user as proxy for fatigue in the first user and determining a resistance level of the first exercise resistance controller of the first exercise machine for a remaining range of motion of the exercise in order to enable the first user to complete a range of motion; analyzing the real-time data with respect to the second user as proxy for fatigue in the second user and determining a resistance level of the second exercise resistance controller of the second exercise machine for a remaining range of motion of the exercise in order to enable the second user to complete the range of motion; automatically adjusting the first exercise resistance controller of the first exercise machine within the three-dimensional (3D) geospatial motion of the first user and across time of the exercise, wherein the first exercise resistance controller adjusts a first resistance force throughout a specified range of motion of the exercise; and automatically adjusting the second exercise resistance controller of the second exercise machine within the three-dimensional (3D) geospatial motion of the second user and across time of the exercise, wherein the second exercise resistance controller adjusts a second resistance force throughout the specified range of motion of the exercise, and wherein the step of automatically adjusting the first exercise resistance controller of the first exercise machine within the three-dimensional (3D) geospatial motion of the first user and across time of the exercise comprises updating a resistance profile that is generated using settings of a space-variations step function, wherein the space-variations defines how a resistance value of the first exercise resistance controller changes dynamically as a cable of the first exercise machine moves through the three-dimensional (3D) plane, wherein the space-variations step functions uses a step function that uses its shape to determine a direction of the resistive force over one or more duration intervals and repetitions.

### BRIEF DESCRIPTION OF THE DRAWINGS

FIG. 1 illustrates the basic structure of an algorithm to unify different sources of data in order to optimize the resistance force to be applied to a training equipment, according to some embodiments.

FIG. 2 illustrates an example data-retrieving layer that is used by the structure described in FIG. 1, according to some embodiments.

FIG. 3 illustrates an example work parameters flow, according to some embodiments.

FIG. 4 illustrates the definition of adaptive resistance blocks throughout the WPS through an event-driven engine that computes an input according to three main elements time-driven slot type , space driven slot type and conditional-driven slot variations, according to some embodiments

FIG. 5 illustrates the different typologies of functions of the resistance profile variation depending on the input from the event driven engine, according to some embodiments.

FIG. 6 illustrates a practical set of examples of combinations of parameters in the event-driven engine that will influence the RPV according to some embodiments.

FIG. 7 illustrates example conditional variations, according to some embodiments.

FIG. 8 depicts an exemplary computing system that can be configured to perform any one of the processes provided herein.

FIG. 9 is a block diagram of a sample-computing environment that can be utilized to implement various embodiments.

FIG. 10 illustrates an example, in flow-chart format, of a system to optimize the force in an exercise and control a possible machine that replaces analog resistance in a training equipment (such as weights or flywheels) with a digitally driven one (such as an electromagnetic engine), according to some embodiments.

The Figures described above are a representative set, and are not exhaustive with respect to embodying the invention.

### DESCRIPTION

Disclosed are a system, method, and article of method and system optimizing and personalizing resistance force in an exercise. The following description is presented to enable a person of ordinary skill in the art to make and use the various embodiments. Descriptions of specific devices, techniques, and applications are provided only as examples. Various modifications to the examples described herein can be readily apparent to those of ordinary skill in the art, and the general principles defined herein may be applied to other examples and applications without departing from the spirit and scope of the various embodiments.

Reference throughout this specification to ‘one embodiment,’ ‘an embodiment,’ ‘one example,’ or similar language means that a particular feature, structure, or characteristic described in connection with the embodiment is included in at least one embodiment of the present invention. Thus, appearances of the phrases ‘in one embodiment,’ ‘in an embodiment,’ and similar language throughout this specification may, but do not necessarily, all refer to the same embodiment.

Furthermore, the described features, structures, or characteristics of the invention may be combined in any suitable manner in one or more embodiments. In the following description, numerous specific details are provided, such as examples of programming, software modules, user selections, network transactions, database queries, database structures, hardware modules, hardware circuits, hardware chips, etc., to provide a thorough understanding of embodiments of the invention. One skilled in the relevant art can recognize, however, that the invention may be practiced without one or more of the specific details, or with other methods, components, materials, and so forth. In other instances, well-known structures, materials, or operations are not shown or described in detail to avoid obscuring aspects of the invention.

The schematic flow chart diagrams included herein are generally set forth as logical flow chart diagrams. As such, the depicted order and labeled steps are indicative of one embodiment of the presented method. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagrams, they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

Definitions

Example definitions for some embodiments are now provided.

Accelerometer is a device that measures proper acceleration (“g-force”). Proper acceleration is not the same as coordinate acceleration (rate of change of velocity).

Accommodating resistance can be fluctuations in muscular force throughout the ROM (see infra) are matched by an equal counterforce.

Activity tracker is a device or application for monitoring and tracking fitness-related metrics such as distance walked or run, calorie consumption, and in some cases heartbeat, muscle exertion and quality of sleep. The term is now primarily used for dedicated electronic monitoring devices that are synced, in many cases wirelessly, to a computer or smartphone for long-term data tracking, an example of wearable technology.

Application programming interface (API) can specify how software components of various systems interact with each other.

Cloud Computing can involve deploying groups of remote servers and/or software networks that allow centralized data storage and online access to computer services or resources. These groups of remote servers and/or software networks can be a collection of remote computing services.

Concentric dynamic contractions can be the shortening phase of a lift, often called the positive aspect. Examples can include rising out of the bottom of a squat, pressing the bar up when benching and standing up with a deadlift.

Eccentric dynamic contractions can occur when the muscles produce a braking force to decelerate rapidly moving body segments or to resist gravity (e.g. slowly lowering barbell). The muscle exerts tension while lengthening.

Dynamic contractions can be contractions with a visible joint movement. JSON is an open-standard format that uses human-readable text to transmit data objects consisting of attribute-value pairs.

Machine learning can use pattern recognition and computational learning theory in artificial intelligence to construct algorithms that can learn from and make predictions on data.

Mobile device can be a computing device that has an operating system (OS) that can run various types of application software. A mobile device can be equipped with Wi-Fi, Bluetooth, NFC, and GPS capabilities that can allow connections to the Internet and other devices, such as an automobile or can be used to provide location-based services. A camera or media player feature for video or music files can also be typically found on these devices along with a stable battery power source such as a lithium battery. A mobile device can also contain sensors like accelerometers, compasses, magnetometers, or gyroscopes, allowing detection of orientation and motion.

Muscular strength is defined as the ability of a muscle group to develop maximal contractile force against a resistance in a single contraction. The force generated by a muscle or muscle group, however, is highly dependent on the velocity of movement. Maximal force is produced when the limb is not rotating (e.g. zero velocity). As the speed of joint rotation increases, the muscular force decreases. For example, strength for dynamic movements is defined as the maximal force generated in a single contraction at a specified velocity. Maximum strength is measured in either Maximum voluntary isometric contraction (kg or N—for static testing), one repetition maximum (or 1-RM) (lbs. or kg—for dynamic testing) and peak torque (Nm—for isokinetic and omnikinetic testing)

Muscular endurance is the ability of a muscle group to exert submaximal force for extended periods. Repetition maximum (RM) can be the maximum weight that the person can lift for a given number of repetitions of an exercise (e.g., eight (8)-RM equals the maximum weight that the person can lift for eight (8) repetitions).

Neural Network (e.g. an artificial neural network) can be a family of models inspired by biological neural networks which are used to estimate or approximate functions that can depend on a large number of inputs and are generally unknown. Artificial neural networks are generally presented as systems of interconnected “neurons” which exchange messages between each other. The connections have numeric weights that can be tuned based on experience, making neural nets adaptive to inputs and capable of learning.

Range of motion (ROM) refers to the distance and direction a joint can move between the flexed position and the extended position.

Repetition is considered the completion of a movement throughout the possible range of motion or a predetermined subset of the possible range within an exercise.

Periodization can include the systematic variation of the intensity and volume of resistance training. The goal of periodization can include, inter alia: (1) to maximize the response of the neuromuscular system (e.g., gains in strength, endurance, power, and hypertrophy) by systematically changing the training or exercise stimulus and (2) to minimize overtraining and injury by planning rest and recovery. The training stimulus may be varied by manipulations in one or more of the following program elements.

Relative strength can be calculated by dividing the 1-RM or Peak Torque values by the user's body mass.

Set Training volume can be the total amount of weight lifted during the work and is calculated by the integral of the weight equivalent lifted throughout repetitions, and sets for each exercise.

Static or Isometric contraction can be muscle contraction with an immovable resistance.

Training volume can include the number of sets, repetitions, or exercises performed by a user.

Training intensity can include the amount of resistance in an exercise.

Wearable technology can include clothing and accessories incorporating computer and advanced electronic technologies. The designs often incorporate practical functions and features. Wearable devices can be embedded with electronics, software, sensors, and connectivity to enable objects to exchange data with a manufacturer, operator and/or other connected devices, without requiring human intervention.

### Example Methods and Algorithms

An algorithm for optimizing the amount of force that should be used during a work session. The algorithm has been designed to work on various resistance engines/systems (e.g. a computerized-exercise resistance controller that is integrated into an exercise device, exercise-machine engines and/or systems as provided in the U.S. Provisional Application No. 62/338,938 filed on 19 May 2016 and titled Method And System Of Optimizing Resistance Force In An Exercise and its Appendices which are incorporated by reference herein, etc.) that can be mounted on a training machine with digital adjustable resistance.

The algorithm can include different layers of data processing that permit the determination of the exact force to be applied to the machine (expressed as a weight-equivalent e.g. in kg. or lbs.). This force is calculated from the elaboration of various information gathered both from the machine, from the user and from population statistics. The algorithm is based on both static and dynamic components, as well as machine learning technologies.

Example Methods and Algorithms

An algorithm for optimizing the amount of force that should be used during a work session. The algorithm has been designed to work on various resistance engines/systems (e.g. a computerized-exercise resistance controller that is integrated into an exercise device, etc.) that can be mounted on a training machine with digital adjustable resistance.

The algorithm can include different layers of data processing that permit the determination of the exact weight-equivalent (e.g. a ‘force’ expressed in Kgs. or Lbs.', etc.) to be applied to the machine. This force is calculated from the elaboration of various information gathered both from the machine, from the user and from population statistics. The algorithm is based on both static and dynamic components, as well as machine learning technologies.

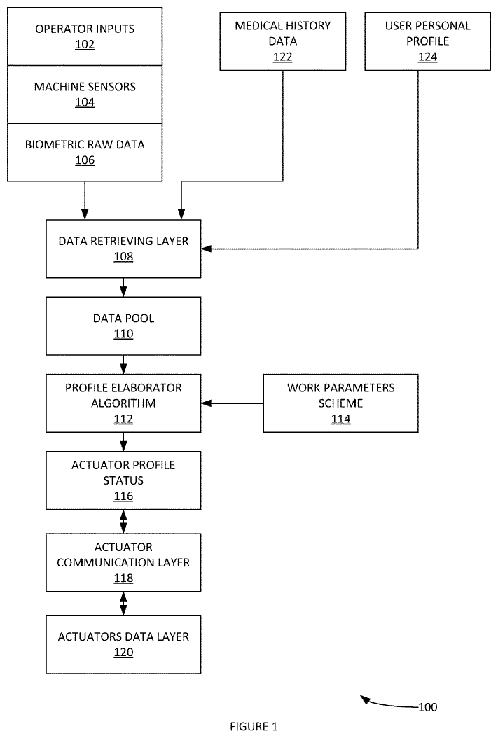

FIG. 1 illustrates an example process 100 for optimizing resistance force in an exercise, according to some embodiments. Process 100 can obtain a user's profile data. A user's profile data can include, inter alia: a user's medical history 232, a user's personal profile, exercise history, etc. Process 100 can obtain real-time data. Real-time data can include, inter alia: operator inputs 702, machine-sensor data 104 (e.g. from accelerometer data, force sensors, etc. placed on the actuator, the body of the machine, the cables, and the handles inter alia.), biometric raw data, etc. Process 100 can implement various mobile device operations as listed in FIG. 1. For example, information from these operations can be provided to an exercise resistance controller.

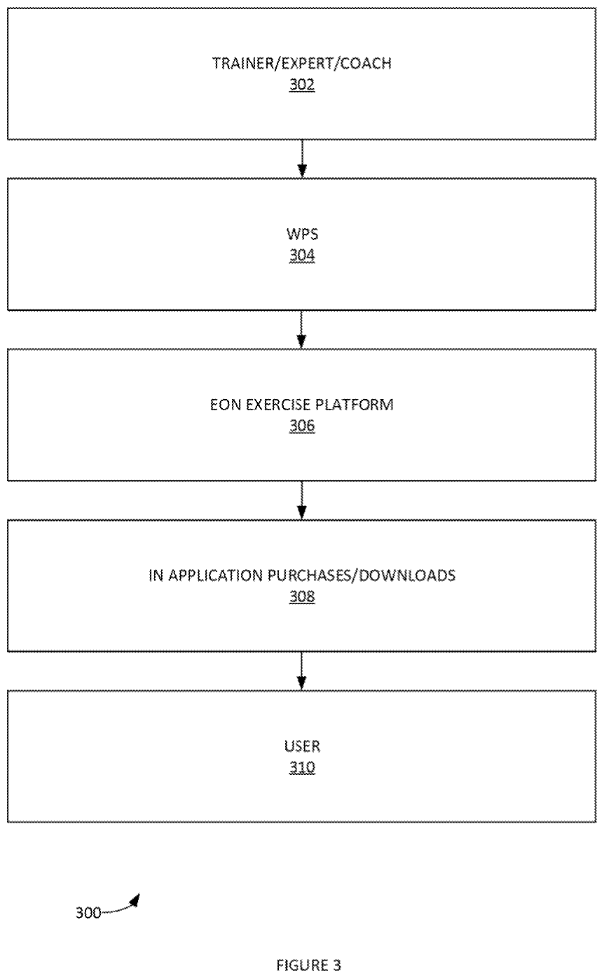

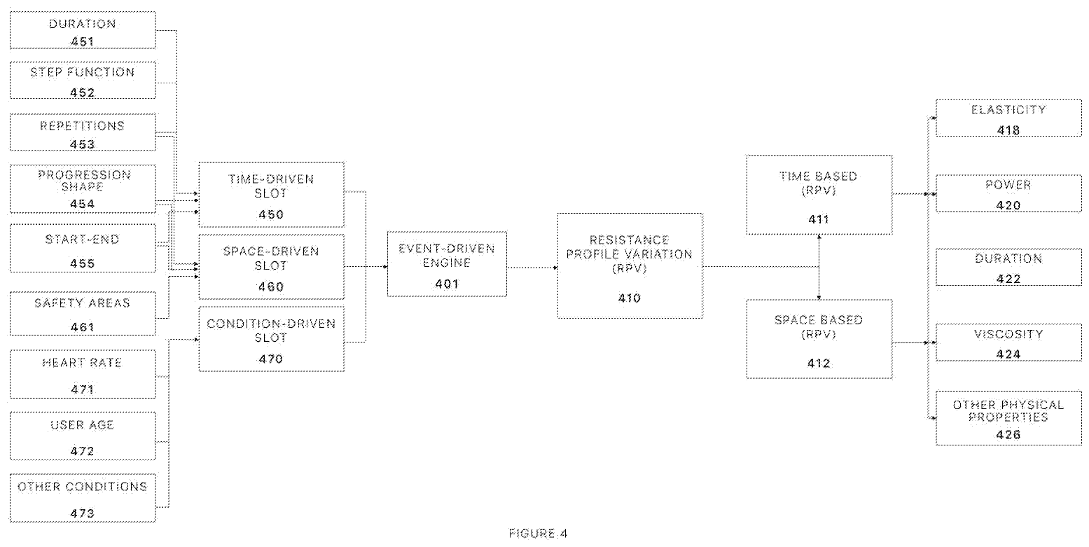

Work parameter scheme 114 (and/or WPS 304 of FIG. 3 infra) can be a cloud-based development environment used to program exercises in blocks according to rules defined in FIGS. 4-7.

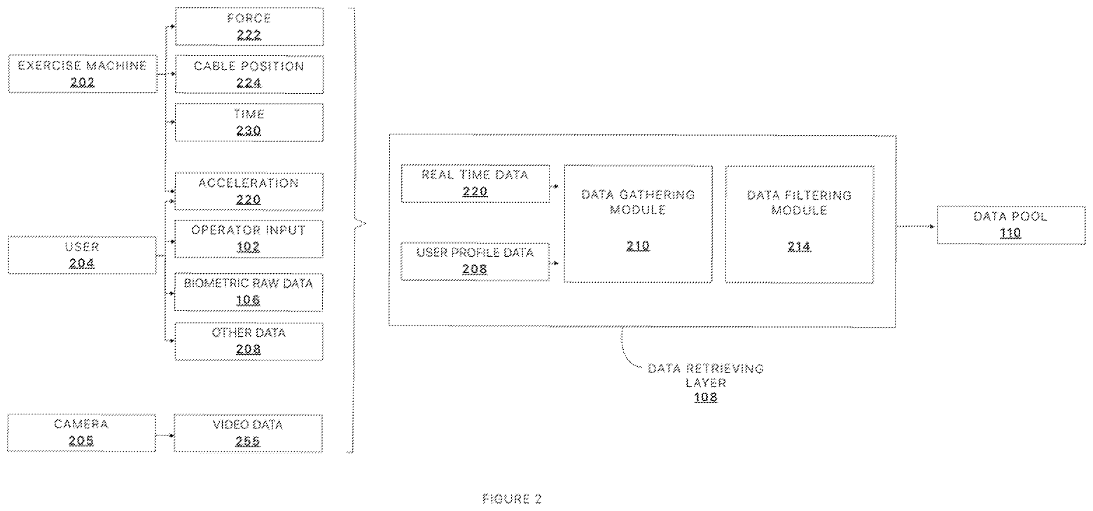

FIG. 2 illustrates an example data-retrieving layer 108, according to some embodiments. The data-retrieving layer (DRL) 108 can be a module that manages operations regarding the acquisition, filtering, storage of the data into the data pool DP 110. The various elements of DRL 108 are now discussed. These data elements can represent real time data (RTD) 206. For example, the can be the information gathered in real time during the session or exercise (e.g. data is produced both by the machine and the user 204 during a training session, etc.) or user profile data (UPD) 208 (e.g. information gathered outside of the session or exercise).

Acceleration data 220 can be obtained. Acceleration data can be acquired from the exercise machine 202 that produces the information or from a user's sensor through BRD 106. Acceleration data can represent the speed increase or decrease at which the cable is pulled by the user 204. Acceleration data can be measured in meters/second\hat{ }2 or any other dimensional equivalent. Speed data can be obtained. Speed data can be acquired from the exercise machine 202 that produces the information. Speed data can represent the speed at which the cable is pulled by the user 204. Speed data can be measured in meters/second.

Force data 222 can be obtained. Force data can be acquired from the machine that produces it (e.g. an exercise machine with integrated force sensors). Force data can represent the force that the user 204 is currently applying to the cable on the machine. Force data can be measured in Newtons and is expressed for a simplicity as kg equivalents herein.

Cable position data 224 can be obtained. Cable position data can be acquired or inferred from a machine that includes a cable or any exercise machine with integrated geospatial or position sensors). Cable position data can represent the relative position of the cable from the starting position (and/or release position). Cable position data can be measured in centimeters (cm) across a two or three-dimensional plane.

Time data 230 can be obtained by a digital time keeping device within the machine (or external to it).

A camera 205 can be used either from inside the machine or as a 3rd party external apparatus in order to capture video data 255 throughout the exercise session. Video data can be stored and interpreted through machine learning and computer vision algorithms. Operator input 102 data can be obtained. Operator input 102 data can be acquired from the exercise machine 202 and is produced when the operator activates the input panel. The normal functions that are usable in the input panel are “increase %” and “decrease %” and absolute force (in Kg-equivalent). Biometric raw data (BRD) 106 can be obtained. BRD 106 can be produced by sensors applied on the user 204 that is performing the exercise. BRD 106 sensors can be of multiple types and measure various aspects of the user's physical state. BRD 106 type can be elaborated into a specific data model developed following the Human Model State (HMS) standard (e.g. see infra).

User profiled data (UPD) 208 data can be obtained. UPD 208 data type can represent information gathered outside of the exercise context. Various types of information can be included in UPD 208. UPD 208 can be related either to the user or to one or more population groups that the user belongs to (e.g. by age, country, etc.).

Medical History Data (MHD) 232 can be obtained. For example, the documented medical history of the user can be included in the MHD 232. MHD 232 can be inserted manually by a physician or can be gathered from specific medical databases (e.g. Microsoft HealthVault®, Apple HealthKit®, etc.).

User Personal Profile (UPP) 234 data can be obtained. UPP 234 data can be autonomously defined by the user. UPP 234 data can be generic and definable. Example UPP 234 data can include, inter alia: age, race, user weight, user height, etc.

User Usage History (UUH) 236 data can be obtained. UUH can be obtained from historical utilization of the exercise devices and algorithms for optimization exercise force such as process 100. Supplemental health/nutrition data (SupD) can be obtained.

Process 100 can connect with various third-party applications that track the user's health/fitness/nutrition. Third party data can be cross-correlated with user performance data to analyze impact of different exogenous factors on overall exercise effectiveness and can be captured via API link after user consent. For example, sample data and related data-providers could be, inter alia: sleep quality (eightsleep.com, sleepnumber.com, etc.); nutrition data (e.g. MyFitnessPal®, etc.); activity trackers (e.g. Fitbit®, jawbone®, misfit®, etc.), etc.

Genotype and/or Genome data (GD) can be obtained. Genotype and/or genome data can be used to determine multiple optimal training, nutrition, and recovery regimen. It is noted that functional SNPs in promoters and regulatory regions can either affect gene expression or change the amino acid sequence of a protein or, in an extreme case, replace an amino acid with a premature stop codon so that the resultant protein is incomplete and maybe non-functional. By accessing data provided by the user from Genotype or Genetic providers, process 100 can adapt training, recovery, and supplementation data to provide optimized performance.

A user's social network data (SND) can be obtained. SND can be used to determine connections between different users and compare UUH data amongst the single users and/or groups of users and by posting UUH data mapped into specific Key performance indicator (KPIs) and graphs onto different social networks such as Facebook or twitter.

Machine learning data (MLD) can be obtained. MLD can consist of statistical analysis of all of the above data-points. It can be especially focused on correlation analysis between UUH, GD and SupD. Population statistics information (Pop_Stat) can be obtained. MLD can also use video data 205 in order to perform human posture analysis and inform the WPS of any needed changes to the Resistance Profile Variation when the posture analysis does not conform with the predefined form evolution during an exercise. MLD can also be used to infer factors specific to the user such as fatigue leveraging a time-series of response data to a certain type of inputs. The MHD 232 and/or UPP 234, as well as the data coming from the force controlling engines and sensors in the exercise device can be obtained anonymously and analyzed to produce population statistics. The data is managed on a secured ‘big data’-type cloud server. Accordingly, process 100 can then create managed profiles that can be used to optimize the algorithms through machine learning processes.

Data gathering and filtering operations can be implemented by data gathering 210 and data filtering module 214. For example, data can be processed before being used by a data filtering module 214 that permits to eliminate noise and inconsistent information. A Data Pool (DP) 110 can be implemented. The DP 110 can store and provide available information to the PEA. This information can be updated by the DRL.

FIG. 3 illustrates an example work parameters flow 300, according to some embodiments. In one example, once the data has been gathered and placed into DP 110, it can be analyzed by the flow 300 in order to determine the force for the exercise. Flow 300 can receive a parameter scheme (WPS) 304 to adapt the machine response to a particular physical situation in a work session. In some examples, the WPS 304 may be optional and flow 300 can operate also without external parameters. When it is included, the WPS 304 enables various parties (e.g. trainers 302, doctors, athletes, etc.) to develop personalized algorithms for various user(s) 310. The WPS Exercise platform 306 is a database that contains all the groups and sequences of exercises that have been developed and saved using the WPS from trainers/experts/coaches and are available to be used on the actuator. Development tools can be provided to create a framework of advanced work sessions that can be downloaded and/or sold (e.g. as in application purchases or downloads 308, etc.).

FIG. 4 illustrates the definition of adaptive resistance blocks throughout the WPS 304 through an event-driven engine 401 that computes an input according to three main elements time-driven slot type 450, space driven slot type 460 and conditional-driven slot variations 470, according to some embodiments. These three inputs are calculated on a millisecond-by-millisecond basis and can independently or conjointly affect the variation of the resistance profile 410

The time-driven slot 450 measures a difference (delta Time or dT) in time across the range of the exercise and determines when in time a change to the resistance profile 410 should occur.

This can be further broken down into discrete ways of calculating dT through variables such as exercise duration 451, step functions 452, repetitions 453, the shape of the time progression of the exercise 454 and start-end of the resistance in time 455.

The space-driven slot 460 measures a difference (delta Space or dS) in space across a space vector calculated from an origin point on a three-dimensional plane and determines at which point in space or through the movement through space a change in the resistance profile 410 should occur. This can be applied by using Real Time Data (RTD) 206 from a cable position and/or other geospatial sensors placed and can be further broken down into discrete ways of calculating dS by considering the progression shape of the exercise across space 454, the start-to-end progression of the exercise across space 425 and the movement across or beyond particular predetermined safety areas 426

A combination of time-driven slot 450 and space-driven slot 460 results in a calculation across four dimensions in the event-driven engine 401 which then also takes into consideration other condition-driven variables that elevate the dimensionality of the calculation.

The condition-driven slot 470 measures triggers across a set of variables that are not defined by time or space but can influence a change to the resistance profile 410. These can encompass sensor data such as Heart Rate 441, user profile information such as user age 442 or other conditions 444 stemming from BRD 106, UPD 208, UPP, UUH, GD, SND and/or MLD.

Once the event-driven engine 401 has calculated the combination of all the inputs it will pass on an output to determine how the resistance profile should change across time 411 and/or space 412. Finally the output gives a set of different possibilities in the quality of the resistance change that encompass elasticity 418, power 420, duration 422, viscosity 424, and other physical properties 426.

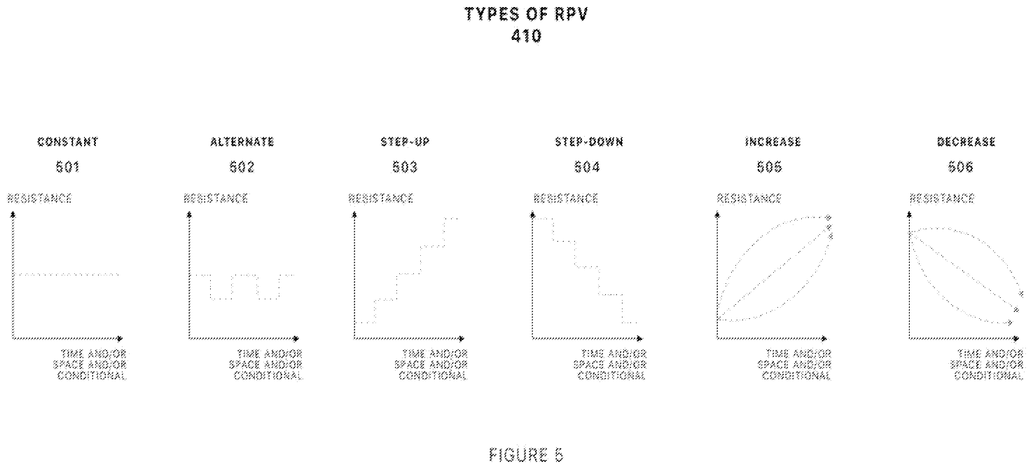

FIG. 5 illustrates the different typologies of functions of the resistance profile variation 410 depending on the input from the event driven engine 401, according to some embodiments. The graphs are simplified versions as the x-axis represents a multi-dimensional set of variables that feed the event-driven engine either singularly or conjointly. They do represent the application of a mathematical formula that applies the parameters described in 418-426 to the resistance of the cable through the actuator.

These functions determine if a resistance will stay constant 501, alternate 502, increase step-wise 503, decrease step-wise 504, increase continuously (e.g. either linearly or according to different mathematical functions) 505, or decrease continuously (e.g. either linearly or according to different mathematical functions) 506.

These functions can be put in sequence and react to the output of the event-driven engine on a millisecond-by-millisecond basis.

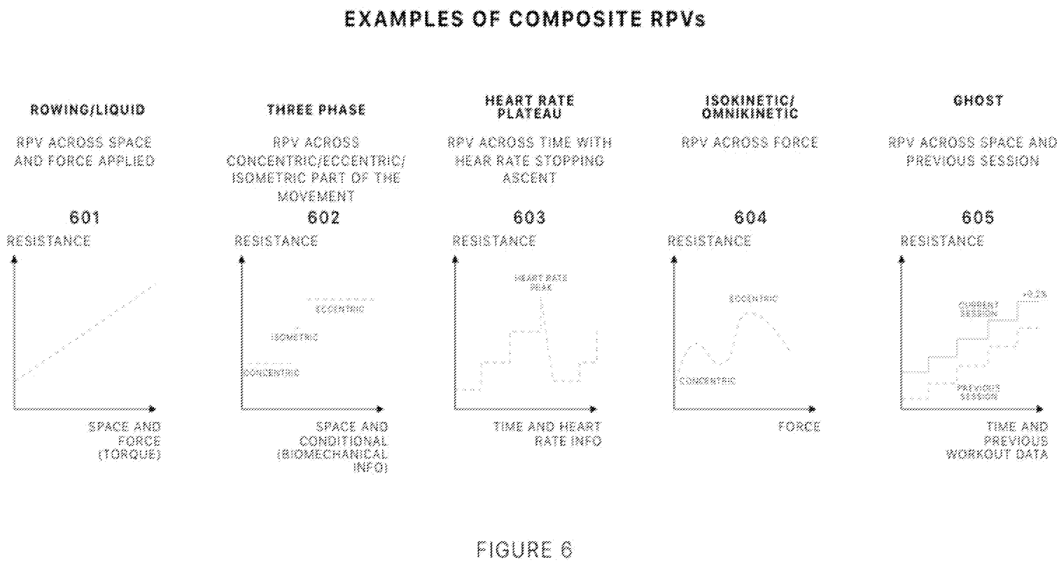

FIG. 6 illustrates a practical set of examples of combinations of parameters in the event-driven engine that will influence the RPV 410 according to some embodiments. Rowing/liquid 601, represents the change in force according to the space parameter and a condition parameter (e.g. torque). In this example the actuator can increase the resistance by a specified amount depending on the amount of movement in space and by the rate of this movement (e.g., the torque). It is thus possible to simulate a rowing environment on water or other conditions not found in nature. Three phase 602, showcases the combination of movement across space and biomechanical information from the UPP or other sources of data that can define the length of a limb to calculate at what point to instruct the actuator to apply stronger resistive force in order to stress the muscle in the most biomechanically optimal fashion. Heart rate plateau 603, showcases the combination of the time variable which instructs the actuator to raise the resistance in a stepwise manner until a certain threshold of the heart-rate is reached which makes the stepwise ascent reset at a lower resistive force. Isokinetic 604 showcases an example where information about force produced by the user is collected by the event-driven engine which will instruct the RPV to almost match it. A further example is provided with the Ghost 605 RPV which can vary in a stepwise fashion according to time and the conditional variable about the previous work profile and instruct the actuator to produce a resistance profile 0.2% higher than the previous session.

The elasticity rule 418 can define a relationship between the extension of the cable and the applied force. The elasticity rule 418 can be considered as an extension to the natural mechanical elasticity of the cable according to some embodiments. The normal elasticity constant k can be increased or decreased in order to emulate a different resulting force that can be obtained if the cable was made of a different material. This rule can permit to emulate the elasticity of different materials for the cable. In some instances, it is a virtual equivalent to the spring constant or force constant present in any mechanical spring of a given material.

The viscosity rule 424 can define a relationship between the speed with which the cable is displaced and the applied force. The viscosity rule 424 can be considered as an extension to the natural viscosity of the cable in the air according to some embodiments. The normal viscosity μ is increased or decreased in order to emulate a different resulting force that can be obtained with a different geometry immersed in a different material/liquid. The viscosity rule can emulate any geometry immersed in any liquid resulting in any viscosity equivalent.

The direction rule 422 can create a relationship between the directional movement of the cable and the force exerted according to some embodiments. Three different states can be defined in this rule. Positive when the cable is pulled in the direction of the user, negative when the cable is released in the direction of the machine and the hold position when the cable is virtually not moved in any direction. Each state can be assigned to a different applied force.

The power rule 420 can define the relationship between the absolute position of the cable and the total force that is applicable to the cable according to some embodiments. This rule can be used to define safety limits to the maximum and minimum force applied to the cable. It is noted that various other rules 426 can be defined to emulate or reproduce normal physical phenomena.

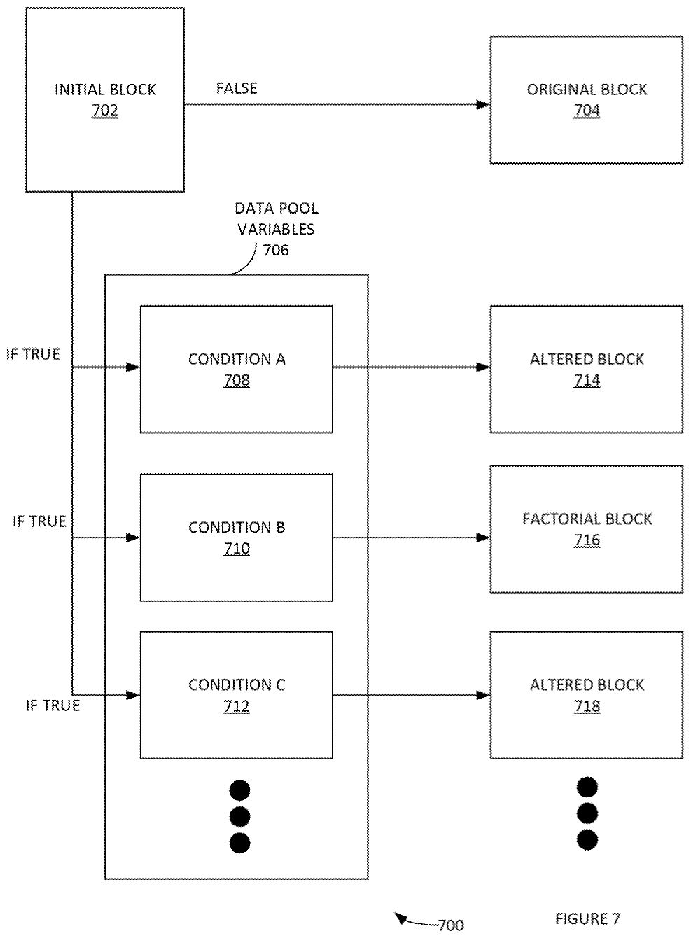

FIG. 7 illustrates example conditional variations, according to some embodiments. Resistance Profile Variations 410 can allow exercise-machine resistance to adaptively change throughout a block. A set of conditions can be specified as data pool variables 706. Data pool variables 706 can obtain variables from DP 110. Data pool variables 706 can be either constant (e.g. age, sex, height, etc.) and/or Real Time (e.g. velocity, heart rate, etc.) (or a combination of these). If a specific variable is TRUE, as shown in FIG. 7, the block is branched off from the original block to create a new block that can be applied by the Parameter Elaborator Algorithm (PEA) 112. This can be either a brand-new block (e.g. altered blocks 714 and 718) that redefines time and space variations or factorial block 716. Factorial block(s) 716 can imply that the profile defined in the time slot is multiplied by a factor that can increase or decrease its resistance.

Returning to FIG. 1, Actuator Profile Status (APS) 116 can be provided. The APS 116 can be a data layer that is sent to the actuator (e.g. in an exercise resistance controller device, etc.) in order to execute the result of the exercise control processes provided herein. This can be defined using the JSON format or other formats. Process 100 can be used to optimize the amount of force that is applied on the exercise-device cable and therefore on each muscle. An optimal determination of this value can be used to prevent an accident caused by an error in the determination of the right amount of weight. This function is particularly important for athlete training and for physical rehabilitation.

For example, process 100 can be used to manage muscle balance. Muscle strength is important for joint stability; however, a strength imbalance between opposing muscle groups (e.g., quadriceps femoris and hamstrings) may compromise joint stability and increase the risk of musculoskeletal injury.

Process 100 can be implemented to reduce overtraining in an athlete user. For example, performing too much total work overtime can stress the body and can lead to overtraining. By using cross-correlation analysis across UUH 236, BRD 106, GenD and SupD, process 100 can increase the accuracy of recovery time and supplementation recommendations to minimize overtraining.

Actuator Communication Layer (ACL) 118 can be provided. ACL 118 can define a communication protocol that permits the algorithm to push the APS 116 to the machine. The ACL does not retrieve any data (e.g. this function can be delegated to the DRL). The ACL 118 can communicate the APS 116 to the exercise-resistance controller device. In one example, the exercise-resistance controller device can return information to the ACL 118 information about the execution of the APS 116 on the machine.

Process 100 can also implement various user genetic-based optimizations. For example, genes can have a major effect on muscle performance. By uploading genomic or genetic data (GD) of large populations of users into the DP 110 cross-referencing their UUH and SupD via the MLD the algorithm can research correlation between different types of genes and performance. This can be applied to genes that have already been highlighted in existing research and to discover new alleles that might be conducive to performance. For example, given the existing research on genetic predisposition process 100 can be used to give suggestions on optimal work type (e.g. sequence of isometric exercises, omnikinetic exercises, etc.), a recovery length and/or a supplementation for enhanced performance, growth and/or recovery.

Additional Exemplary Computer Architecture and Systems

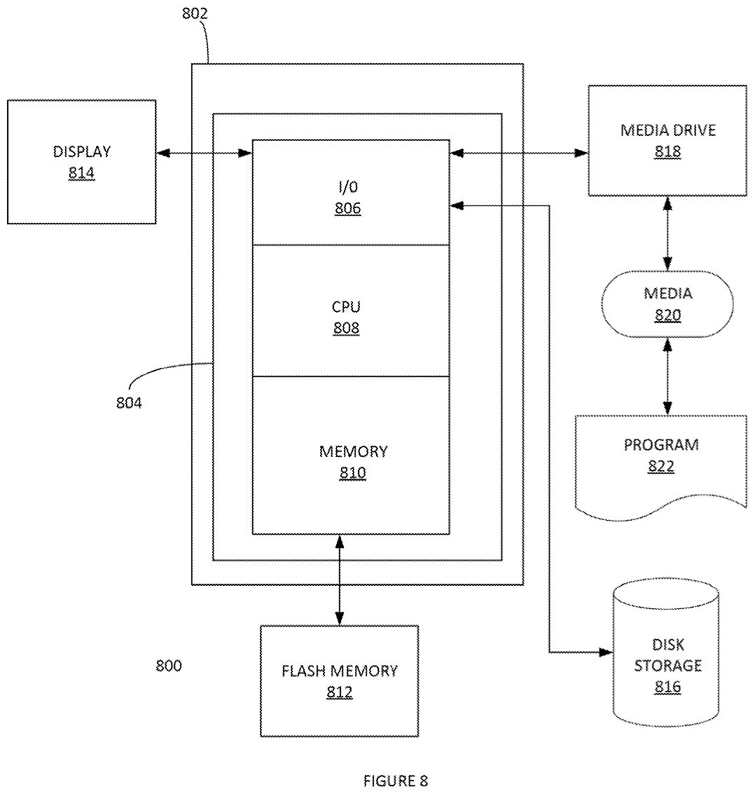

FIG. 8 depicts an exemplary computing system 800 that can be configured to perform any one of the processes provided herein. In this context, computing system 800 may include, for example, a processor, memory, storage, and I/O devices (e.g., monitor, keyboard, disk drive, Internet connection, etc.). However, computing system 800 may include circuitry or other specialized hardware for carrying out some or all aspects of the processes. In some operational settings, computing system 800 may be configured as a system that includes one or more units, each of which is configured to carry out some aspects of the processes either in software, hardware, or some combination thereof.

FIG. 8 depicts computing system 800 with a number of components that may be used to perform any of the processes described herein. The main system 802 includes a motherboard 804 having an I/O section 806, one or more central processing units (CPU) 808, and a memory section 810, which may have a flash memory card 812 related to it. The I/O section 806 can be connected to a display 814, a keyboard and/or other user input (not shown), a disk storage unit 816, and a media drive unit 818. The media drive unit 818 can read/write a computer-readable medium 820, which can contain programs 822 and/or data. Computing system 800 can include a web browser. Moreover, it is noted that computing system 800 can be configured to include additional systems in order to fulfill various functionalities. Computing system 800 can communicate with other computing devices based on various computer communication protocols such a Wi-Fi, Bluetooth° (and/or other standards for exchanging data over short distances includes those using short-wavelength radio transmissions), USB, Ethernet, cellular, an ultrasonic local area communication protocol, etc.

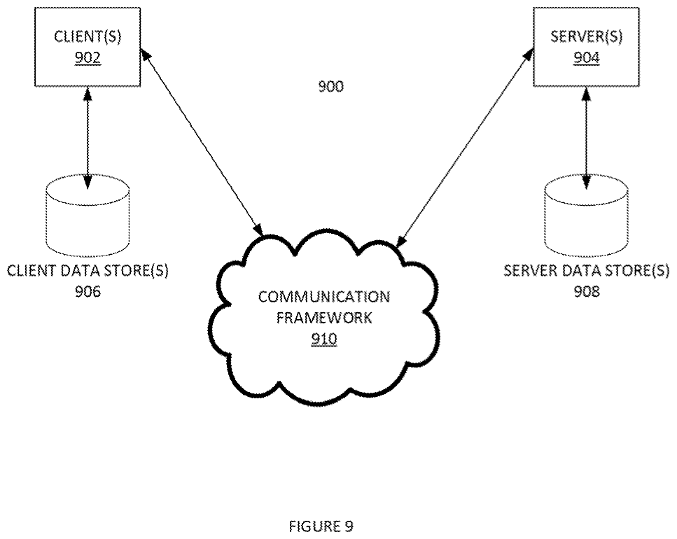

FIG. 9 is a block diagram of a sample-computing environment 900 that can be utilized to implement various embodiments. The system 900 further illustrates a system that includes one or more client(s) 902. The client(s) 902 can be hardware and/or software (e.g., threads, processes, computing devices). The system 900 also includes one or more server(s) 904. The server(s) 904 can also be hardware and/or software (e.g., threads, processes, computing devices). One possible communication between a client 902 and a server 904 may be in the form of a data-packet adapted to be transmitted between two or more computer processes. The system 900 includes a communication framework 910 that can be employed to facilitate communications between the client(s) 902 and the server(s) 904. The client(s) 902 are connected to one or more client data store(s) 906 that can be employed to store information local to the client(s) 902. Similarly, the server(s) 904 are connected to one or more server data store(s) 908 that can be employed to store information local to the server(s) 904. In some embodiments, system 900 can instead be a collection of remote computing services constituting a cloud-computing platform.

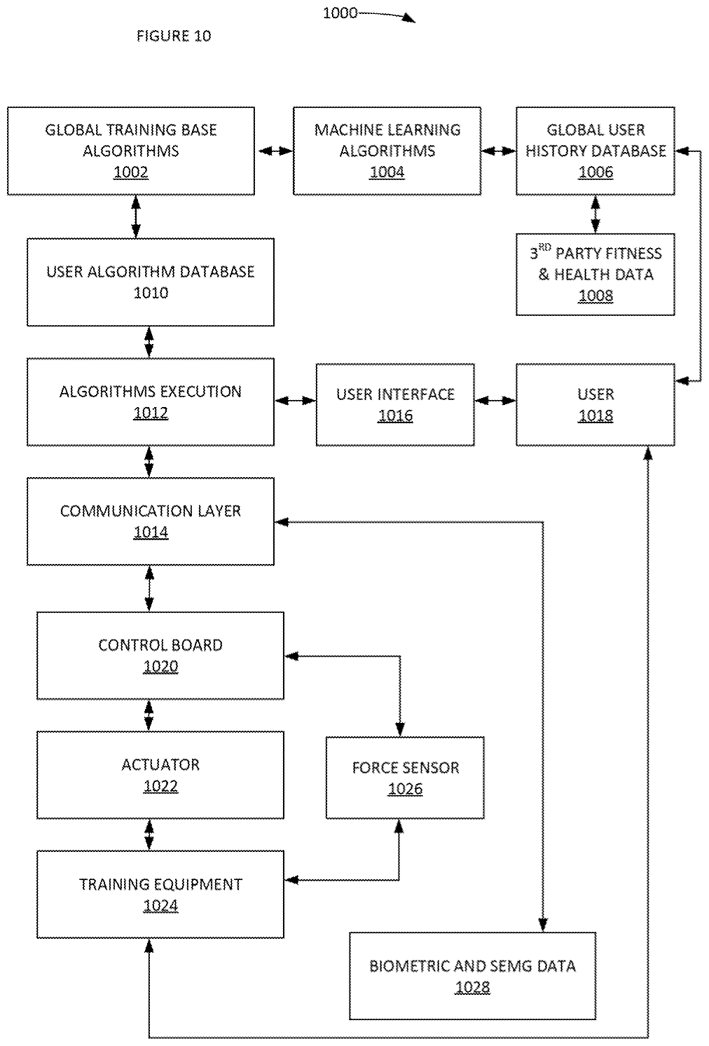

FIG. 10 illustrates an example, in flow-chart format, of a system 1000 to optimize the force in an exercise and control a possible machine that replaces the weights in a training equipment with an engine, according to some embodiments. It effectively showcases the loop between single user data generation during exercise, data aggregation, data elaboration and re-execution. Using this positive feedback loop it is possible to continuously test new exercise regimes and ameliorate exercise quality. Global training base algorithms 1002 can be a database of exercise training algorithms that have been developed using the Work Parameter Scheme 114. Machine learning algorithms 1004 can apply various machine learning algorithms to optimize and/or otherwise improve user exercise experience and/or operation of exercise with training equipment 1024. Global user history database 1006 can be a history of user exercise history that accumulates in the User Personal Profile 234 of any user utilizing the system across time. Third party fitness and health data 1008 can be used to gather supplemental data to enrich the dataset during user exercises and outside of exercise. User algorithm database 1010 is the subset of globally available algorithms on the Eon exercise platform 306 that the user has access to or has purchased 308. Algorithms execution 1012 happens when the user has selected one of the algorithms available in his User algorithm database 1010 for execution on a digital resistance exercise machine. User interface 1016 can enable a user to input data and controls into system 1000. Control board 1020 is a firmware layer that contains the code to execute and monitor processes 112-120. Actuator 1022 is any device that can digitally control resistance such as an electromagnetic engine. Training equipment 1024 can be a piece of either traditional exercise equipment (e.g. a stationary bicycle, a weightlifting machine, etc.) or new types of equipment specially designed to mount Actuators. Force sensor 1026 provides a real-time reading of force being exerted by the user while utilizing the system. It enriches the Machine Sensors 104 dataset and can be placed in different physical spaces such as handle-bars or in the connecting cable. Biometric and sEMG data 1028 provides real-time and non-real-time data from the user (heartbeat, sleep quality, muscle tension) used to optimize resistance levels.

Example Use Cases

One example of the use of this method is the massively parallel automation, optimization and personalization of training lessons streamed to a home and/or to work facilities. Various entities, like Peloton®, stream their exercise content to the user's home. The resistance profiles of their stationary bicycle can be adjusted manually by the user during the exercise. Utilizing this method, one exercise that has been compiled through the WPS 114 can be streamed to millions of homes that have digitally adjustable resistance devices simultaneously with a different resistance profile automatically applied through the conditional variations 414 to every different user that falls within the defined condition. In this way, while doing the same movement on a machine a sixty-year-old lady, a thirty (30) year-old man and a fifteen (15) year old child can be performing the work with different resistance curves that are continuously adjusted throughout the exercise to match the settings defined in the WPS.

An example of automated resistance training is now provided. Different muscle groups have different optimal resistance curves that can stimulate them in the most optimal way. For instance, a bicep curl is best performed with a resistance curve that has the shape of an arc given that it can express the most power in the middle of the range of motion. For instance, using a preconfigured function on the WPS 114 (e.g. space variation type 416), a user can train with an optimized resistance curve that changes resistance in space starting at 20 kg with stretched arms, to reach thirty-five kilograms (35 kg) in the middle and twenty-five kilograms (25 kg) at the end of the curl. Moreover, this resistance could change through different repetitions by defining the resistance progression shape 404. Again, as an example the second repetition could be a ten percent (10%) step up from the previous one so starting at 22 kg, reaching thirty-eight point five kilograms (38.5 kg) in the middle and twenty-seven point five (27.5 kg) at the end of the curl. When the user is a woman then a function defined within conditional variations 414 could automatically apply a reduction in force (e.g. fifty percent (50%)) but follow the same resistance path as the one originally defined.

A further example of automated resistance training is now provided. Specifically, an example of power matching training (e.g. omnikinetic training) is provided. The user can download a power matching training module on a mobile application. This module has a simple objective, to match the force (A1.2) exerted from the machine to the maximum muscle output (potential) on the concentric and the eccentric part of the exercise (or only one of those). This can be applied to several types of exercises currently performed on exercise machines equipped with digital adjustable resistance (DAWs) or more broadly digital adjustable resistance, including for instance leg-extension, lateral-pulldown, or chest fly machines. In this example, a chest fly machine is used.

Before the start of the training, the user selects the power matching exercise from his mobile device and synchronizes with the machine he intends to use. The Parameter Elaboration Algorithm (PEA) can synchronize with pre-loaded conditions from the User-Profile Data (UPD) 208 such as user age, Medical History, and his Historical Usage Data. These data-points are utilized to schedule the duration of the WPS for each exercise. Alternatively, the user or a trainer can determine the duration of the WPS.

As the user starts pulling the grips to start executing the concentric part of the chest fly the PEA utilizes the Biometric Raw Data (BRD 106) feed to set the initial level of force to be applied. Data stemming from the force sensor or simple calculations can provide data on the force that the user is applying to the cable by pulling the grip. In addition to that if the user has ulterior wearable sensor devices attached the BRD 106 can be enriched by Heart Rate data coming from a Fitbit or other wearable and by surface Electromyographical (sEMG) data, coming from athletic apparel such as Athos®. The Actuator can increase the force applied to the cable that is being released by the user pulling force to a level where the user can advance in his chest fly movement slightly. Once the user progresses in this movement the BRD 106 can be collected every five milliseconds in order to inform the PEA and adjust the force applied by the actuator up or down. If the user's force outmatches the actuator force by a degree such that the cable is pulled by more than ten centimeters (10 cm) the actuator can step-up the force. If the user's force is more or less equal to the force of the actuator, then the actuator force can be slightly decreased. If the user's force is less than the force of the actuator, then the actuator force can be decreased more. This algorithm applies throughout the duration of the concentric move and can cease at the end of the concentric chest fly (e.g. defined by one centimeter (1 cm) of cable pulled/height of the user).

If the user has selected eccentric training, a similar paradigm can be applied to the exercise but in reverse. In the chest fly example, eccentric training starts with the user holding the grips far from his chest and resisting the force being applied by the cable being pulled back. Just as in the concentric example, the BRD 106 data can inform the PEA and lead to continuous adjustment of the actuator force. In this case though the actuator can react in the opposite way by decreasing the force of the cable pull in case force user<force actuator and increase if force user>force actuator.

An example of a virtual spotter is now provided. Within the WPS 114, it is possible to define a function that acts as what is commonly referred to as a spotter in the fitness vernacular. A spotter can act to assist a person during a training session by effectively lowering the overall level of resistance born by the current lifter. It is noted that, due to fatigue, the person exercising reaches muscular failure and cannot complete the required range of motion in the exercise. By analyzing specific data points that act as proxy for fatigue (e.g. acceleration from machine sensors 104, heart rate from BRD 106, etc.) the right time to alleviate resistance can be determined and implemented in order to enable the user to complete the range of motion. If acceleration diminishes below a threshold such as one centimeter (1) cm/s then the function within the WPS 114 can diminish the resistance by a level set by the trainer and/or inferred through machine learning algorithms to be optimal. For instance, with a bicep curl exercise example provided herein, if the user during the fifth repetition is stuck at ninety-three degrees (93°) of their range of motion then, the virtual spotter can diminish the resistance by seven percent (7%) for the remaining set of repetitions the user wishes to finish.

An example of resistance assisted posture correction is now provided. Leveraging algorithms developed through MLD or by using third party algorithms such as YOLO or R-CNNs video data 255 can be used to assess if a person is using the correct posture throughout an exercise. Pairing RTD such as acceleration 220, force 222 and cable position 224 with visual data can further enhance the precision of these algorithms. In the case that the event-driven engine is notified of a discrepancy between the optimal posture and the one the user is adopting throughout the exercise the resistance can be adjusted in order to safe-guard the user's musculoskeletal system or to nudge the user that the posture is incorrect. This can be paired as well with visual or audio signals.

An example of automated cardio zone training is now provided. BRD 106 can be enriched by capturing heart rate data through tracking devices such as the ones included in the Apple iWatch or Polar chest straps or through exercise handles that have integrated sensors. During training, the optimal range of cardiac training varies from person to person and on a general basis follows a declining curve that varies with age. Within the WPS 114 a conditional rule 414 can be established to change the resistance levels in exercise based on reaching a specific heart rate threshold level for different users that have either been predetermined by a trainer or have been elaborated through machine learning leveraging data from historical use and the user personal profile 234 in general and even the medical history data 232. For example, for a male of thirty (30) years of age the average maximum heart rate is one-hundred and ninety (190) beats per minute. While working on a rowing-like motion on a digitally adjustable resistance machine once the heart rate reaches above one hundred and seventy-five (175) beats per minute for more than a minute the resistance level of the exercise is diminished by ten percent (10%). Vice versa if the heart rate reaches below 130 beats per minute for more than a minute then the resistance is increased by ten percent (10%), this adjustment continues for the remaining exercise time specified in the WPS.

An example of breathing assistance is now provided. Muscle resistance can be significantly changed by breathing patterns. For example, it is well-known and accepted that a deep inspiration during weightlifting enables one to a greater lifting potential. Analyzing breathing patterns received through the BRD 106 received by smart wearables such as Athos, the system described by Process 100 system can cross-correlate these with the machine force output and recommend changes to the breathing pattern via mobile handset notification and include breathing pattern data to graphs and analytics.

An example of use of non-training data for training optimization is now used. BRD 106 is not limited to data generated during the exercise itself. A few examples might be heart rate data during the remainder of the day or the night, which if analyzed as heart rate variability data can provide a proxy for overall fitness level during the day and be used to modify the resistance curves or the overall training scheduling. A similar approach could be used when using sleep data in order to better program optimal exercise sequencing and progression as well as provide further information to other values such as fatigue.

An example of sports specific training is now provided. Using geo-spatial motion tracking sensors integrated in the handles or third-party geospatial sensors such as the ones provided by companies, like Enflux®, the resistance could be modified taking into consideration specific geo-spatial areas that the user desires to hit. For example, if a baseball player may wish to simulate a bat hit, the resistance could be adjusted throughout the range of motion to reach a peak level at a specific geo-spatial point predefined.

An example of automated medically personalized training is now provided. Using MHD 232 of a user, which might include information about conditions such as arthritis or rheumatism, using conditional variations 414 in the WPS 114 the training can be adjusted in order to avoid specific pain points by lowering resistance levels that might hurt the muscle groups affected by the specific condition. Other cases, where a condition can affect the optimal heart rate for a user than MHD 232 can inform the WPS to alter the optimal training zone as discussed in paragraph 93. An example exergaming protocol is now provided. The variables produced throughout the repetition/set/sessions can be used to produce interactive graphics that can be then leveraged for intelligence and motivational scopes. The picture below outlines some of the possibilities of benchmarking force production over a specific set and benchmarking it to the previous session (ghost_previous) drawn from the user's UPP 234 and the ones coming from a specific friend (leveraging SND), a set of friends or the other users in the same exercise group (e.g. by plotting data from the friend's exertion specific UPP 234 or from metadata of his peer-group/age-group or other predetermined groups that match the user's UPP 234. In the picture the user can see in real-time how his current exertion level is proceeding in terms of Force Level achieved (e.g. measured by torque, total work or other exercise indicators produced) with the explicit goal of beating his friend or his previous session.

This data can also be used at an aggregate level by fitness studios to launch internal competitions to benchmark top users against each other or to show users that have achieved the greatest improvements throughout the session. It can also be gamified and used by fitness chains to have users of different gyms compete against each other. In one example application, the integral of torque produced over the number of users can be calculated. Various entities (e.g. gyms, users, teams, etc.) can compete on which entity achieves the highest relative number.

In one example, a computing method useful for automating, personalizing, and optimizing a resistance force in an exercise across time and space, includes the step of providing a personalized training lesson for a first user and a second user. The personalized training lesson comprises a work parameter scheme (WPS). The method includes providing a first exercise machine. The first exercise machine comprises a first digitally adjustable resistance device. The method includes providing a second exercise machine. The second exercise machine comprises a second digitally adjustable resistance device. The method includes providing a first biometric sensor coupled with the first user performing an exercise on the first exercise machine. The method includes providing a second biometric sensor coupled with the second user performing an exercise on the second exercise machine. The method includes obtaining a first user's profile data. The first user's profile data comprises a first user's history of exercising on the first exercise machine. The method includes obtaining a second user's profile data. The second user's profile data comprises a second user's history of exercising on the second exercise machine. The method includes obtaining a first user input into a first exercise resistance controller of the first exercise machine; obtaining a second user input into a second exercise resistance controller of the second exercise machine. The method includes, while the first user and the second user perform one or more repetitions of the exercise on the first exercise machine and the second exercise machine, respectively: obtaining a real-time data of a set of parameters of the one or more repetitions of the exercise on the first exercise machine and the second exercise machine. The real-time data is used as a proxy for fatigue in the first user and the second user. The method includes obtaining a first user's biometric data. The method includes obtaining a second user's biometric data; and based on the real-time data, the first user's biometric data, the second user's biometric data, the first user input into the first exercise resistance controller, the second user input into the second exercise resistance controller, the first user's profile data, the second user's profile data, and the WPS. The method includes analyzing the specified data points that act as proxy for fatigue, The specified data points that act as proxy for fatigue comprise an acceleration from a machine sensor and a heart rate from a BRD; analyzing the real-time data with respect to the first user as proxy for fatigue in the first user and determining a resistance level of the first exercise resistance controller of the first exercise machine for a remaining range of motion of the exercise in order to enable the first user to complete a range of motion. The method includes analyzing the real-time data with respect to the second user as proxy for fatigue in the second user and determining a resistance level of the second exercise resistance controller of the second exercise machine for a remaining range of motion of the exercise in order to enable the second user to complete the range of motion. The method includes automatically adjusting the first exercise resistance controller of the first exercise machine within the three-dimensional (3D) geospatial motion of the first user and across time of the exercise. The first exercise resistance controller adjusts a first resistance force throughout a specified range of motion of the exercise. The method includes automatically adjusting the second exercise resistance controller of the second exercise machine within the three-dimensional (3D) geospatial motion of the second user and across time of the exercise. The second exercise resistance controller adjusts a second resistance force throughout the specified range of motion of the exercise. The step of automatically adjusting the first exercise resistance controller of the first exercise machine within the three-dimensional (3D) geospatial motion of the first user and across time of the exercise comprises updating a resistance profile that is generated using settings of a space-variations step function. The space-variations define how a resistance value of the first exercise resistance controller changes dynamically as a cable of the first exercise machine moves through the three-dimensional (3D) plane. The space-variations step function uses a step function that uses its shape to determine a direction of the resistive force over one or more duration intervals and repetitions.

### CONCLUSION

Although the present embodiments have been described with reference to specific example embodiments, various modifications and changes can be made to these embodiments without departing from the broader spirit and scope of the various embodiments. For example, the various devices, modules, etc. described herein can be enabled and operated using hardware circuitry, firmware, software or any combination of hardware, firmware, and software (e.g., embodied in a machine-readable medium).

In addition, it can be appreciated that the various operations, processes, and methods disclosed herein can be embodied in a machine-readable medium and/or a machine accessible medium compatible with a data processing system (e.g., a computer system), and can be performed in any order (e.g., including using means for achieving the various operations). Accordingly, the specification and drawings are to be regarded in an illustrative rather than a restrictive sense. In some embodiments, the machine-readable medium can be a non-transitory form of machine-readable medium.

What is claimed as new and desired to be protected by Letters Patent of the United States is:

1. A computing method useful for automating, personalizing, and optimizing a resistance or a force for human exertion across time and space, the method comprising:
providing a personalized exercise session for a first user and a second user, wherein the personalized exercise session comprises a work parameter scheme (WPS);
providing a first exercise machine, wherein the first exercise machine comprises a first digitally adjustable resistance device;
providing a second exercise machine, wherein the second exercise machine comprises a second digitally adjustable resistance device;
providing a first set of sensor data (biometric or not) coupled with the first user performing an exercise on the first exercise machine;
providing a second set of sensor data (biometric or not) coupled with the second user performing an exercise on the second exercise machine;
obtaining a first user's profile data, wherein the first user's profile data can comprise a first user's history of utilizing the first exercise machine or any other relevant data to determine his physical condition;
obtaining a second user's profile data, wherein the second user's profile data can comprise a second user's history of utilizing the second exercise machine or any other relevant data to determine his physical condition;
obtaining a first user input into a first resistance controller of the first exercise machine;
obtaining a second user input into a second resistance controller of the second exercise machine;
while the first user and the second user perform one or more repetitions of the exercise on the first exercise machine and the second exercise machine, respectively:
obtaining a real-time data of a set of parameters of the one or more repetitions of the exercise on the first exercise machine and the second exercise machine, and utilizing the real-time and user profile data to optimize the exercise for the user according to rules specified in the WPS such as his usage history, biomechanical characteristics, and inferred factors such as fatigue or posture in the first user and the second user;
obtaining a first user's biometric data;
obtaining a second user's biometric data; and
based on the real-time data, the first user's biometric data, the second user's biometric data, the first user input into the first resistance controller, the second user input into the second resistance controller, the first user's profile data, the second user's profile data, and the WPS:
analyzing the specified data points that act as proxy for fatigue, wherein the specified data points that act as proxy for fatigue comprise an acceleration from a machine sensor, a heart rate and other BRD or MLD;
analyzing the real-time data with respect to the first user and determining a resistance level of the first resistance controller of the first exercise machine for a remaining range of motion of the exercise in order to enable the first user to complete a range of motion;
analyzing the real-time data with respect to the second user and determining a resistance level of the second resistance controller of the second exercise machine for a remaining range of motion of the exercise in order to enable the second user to complete the range of motion;
automatically adjusting the first exercise resistance controller of the first exercise machine within the three-dimensional (3D) geospatial motion of the first user and across time of the exercise, wherein the first exercise resistance controller adjusts a first resistance force throughout a specified range of motion of the exercise; and
automatically adjusting the second exercise resistance controller of the second exercise machine within the three-dimensional (3D) geospatial motion of the second user and across time of the exercise, wherein the second exercise resistance controller adjusts a second resistance force throughout the specified range of motion of the exercise, and
wherein the step of automatically adjusting the first exercise resistance controller of the first exercise machine within the three-dimensional (3D) geospatial motion of the first user and across time of the exercise comprises updating a resistance profile that is generated using settings of a space-variations step function, wherein the space-variations defines how a resistance value of the first exercise resistance controller changes dynamically as a cable of the first exercise machine moves through the three-dimensional (3D) plane, wherein the space-variations step functions uses a step function that uses its shape to determine a direction of the resistive force over one or more duration intervals and repetitions.

2. The computerized method of claim 1, wherein the real-time data may comprise a device that is capable of evaluating the time, acceleration, speed, direction, or the absolute position of the first exercise machine or one of its components and the second exercise machine or one of its components.

3. The computerized method of claim 2, wherein the real-time data may comprise biometrical data (e.g. heart rate, temperature, pression) of the first user and the second user.

4. The computerized method of claim 3, wherein the real-time data comprises Medical History data such as arthritis of the first user and second user.

5. The computerized method of claim 4, wherein the real-time data comprises data derived entirely or partially from a Machine Learning process of the first user and second user.
